(19) United States
(12) Patent Application Publication
(10) Pub. No.: US 2024/0120066 A1
(43) Pub. Date: Apr. 11, 2024

(54) INTERACTIVE THERAPY BOOTH

(71) Applicant: Ex-Nihilo (Out of Nothing) Inc., Marietta, GA (US)

(72) Inventor: Gloria Umanah, Cartersville, GA (US)

(21) Appl. No.: 17/938,102

(22) Filed: Oct. 5, 2022

(51) Int. Cl. G16H 20/70; A61B 5/16; A61L 2/24; G06F 3/01; G06F 3/04847; G06V 40/12; G06V 40/18; G16H 40/67

(52) U.S. Cl. CPC G16H 20/70; A61B 5/165; A61L 2/24; G06F 3/013; G06F 3/04847; G06V 40/1365; G06V 40/18; G16H 40/67; A61L 2202/14

(57) ABSTRACT

An interactive therapy booth can be placed outdoors in city areas. The therapy booth can include a shroud that shields a touchscreen from weather. The touchscreen can be included as part of an insert that couples the shroud. The therapy booth can recognize a user looking at the screen. The booth can play an experience clip with an uplifting message, music, and soothing lighting. The experience clip, lighting, or even lightbox therapy can be selected by the booth based on environmental factors, such as day of year and outside weather or temperature. The booth can further collect some user input regarding the user's current mental state, such as whether they are sad, lonely, or hungry. Based on the input, a map can display on the screen with at least one responsive support opportunity identified on the map.

### BACKGROUND

A homeless person living on the streets can go three to six months without even being looked in the eye. Limited tools exist for helping the mental wellbeing of the homeless. Other than shelters, people living on the streets generally have no mechanisms to support mental health. Mental health tools are also lacking in other environments. For example, prisons, hospitals, schools, and even mental health facilities often do not have an easily accessible way for people to receive immediate help.

Mental health problems abound in various segments of the population. Every 40 seconds, someone dies by suicide. With less connection and more division than ever before, people are experience the pain and hopelessness that accompanies feeling unseen. This problem affects individuals, families, and communities everywhere.

All people have an innate desire for connection. People need a sense of dignity and need to feel valued. People deserve to be seen regardless of their stage of life, belief system, demographic, or disability.

Phone booths used to exist in city areas as a means of connection with others. However, with the advent of cellphones, phone booths are a relic of the past. People who live on the streets may not have consistent access to smartphones or computers. Additionally, while libraries and other public buildings may provide some access during operational hours, it may not be apparent how to establish meaningful connection or to seek therapy using those machines. And the machines are not available outside of business hours.

Therefore, a need exists for interactive therapy booths that can be placed in outdoor areas and provide mental health therapy and messages of hope to users.

### SUMMARY

The examples described herein relate to interactive therapy booths. The therapy booth can include a metal shroud with an insert. The insert can include a screen, which can be a touchscreen in some examples. The shroud can have an open front, a closed back, and a depth of six to 18 inches. The insert can be recessed into the shroud such that the screen is protected from weather elements and from direct sunlight during peak brightness hours.

The screen can provide a graphical user interface (“GUI”). The screen can include touchscreen technology, allowing for user interaction with the GUI. The GUI can display video and support options. The GUI screen can be backlit with IPS technology in one example. A message can be below the screen, such as “where no one goes unseen.”

A combination of features is delivered by the therapy booth to synergistically provide therapy to users. The booth can provide a message on top of background music. The message can be any type of words or text, such as creative messages, spoken word, words of encouragement, or affirmative words. The messages can be developed by creatives, therapists, or others. The message can promote ideas related to leaving behind insecurities and realizing that the individual is uniquely who they are meant to be, with their own story of struggles, failures, and victories. Visual animation can be presented on the GUI to enhance the experience for those with hearing disabilities and can also stimulate visual creativity. The message can last about three minutes and continually promote user attention to the message. Alternatively, the message can be shorter.

Voiceover elements can be used in tandem with animation as part of an experience clip or to choose an experience clip. For example, the GUI can prompt the user to participate in breathing exercises to calm the user down or prompt the user to do and say other things. An animated character can help the user through these prompts or exercises.

Eye tracking can promote user eye contact with the screen and can also determine when the user has walked away or is not engaging with the therapy booth. Targeted hyper sound helps effectively maintain the user experience. Since the therapy booth is placed outdoors in city areas, targeted sound can help the user hear the messages and music over street noise. In one example, headphones connected to the therapy booth can optionally be worn by the user.

The therapy booth can also apply light box therapy. Light box therapy can be an effective treatment for those with seasonal depression. The light box therapy can be combined with the spoken word message in an example. The light box component can include at least one bulb that emits 10,000 lux of light or more. In one example, the light box therapy is activated based on the outdoor light conditions or time of year. For example, on an overcast day in fall, the lightbox lamp can provide light that mimics outdoor lighting from spring or summer.

The GUI can display nearby connection points so that the user can engage with community support organizations. For example, the connection points can provide detail on addresses, directions, times, and services provided. This can help ensure that the user knows where they can go for further in person support.

While a server example is explained, a web server is not required in all examples. Instead, the system can operate based on an application executing on a computing device operated by the user, in an example.

It is to be understood that both the foregoing general description and the following detailed description are exemplary and explanatory only and are not restrictive of the embodiments, as claimed.

### BRIEF DESCRIPTION OF THE DRAWINGS

The accompanying drawings, which are incorporated in and constitute a part of this disclosure, illustrate various embodiments and aspects of the present invention. In the drawings:

FIG. 1 illustrates a perspective view of an example therapy booth.

FIG. 2A illustrates a front view of an example therapy booth.

FIG. 2B illustrates a side view of an example therapy booth.

FIG. 3A illustrates a front view of an example insert for a therapy booth.

FIG. 3B illustrates a side view of an example insert for a therapy booth.

FIG. 3C illustrates a top view of an example base plate for a therapy booth.

FIG. 4 is an example flowchart of steps performed by a therapy booth.

FIG. 5 is an exemplary illustration of system components for providing interactive user therapy.

### DESCRIPTION OF THE EXAMPLES

Reference will now be made in detail to the present exemplary examples, including examples illustrated in the accompanying drawings. Wherever possible, the same reference numbers will be used throughout the drawings to refer to the same or like parts. The described examples are non-limiting.

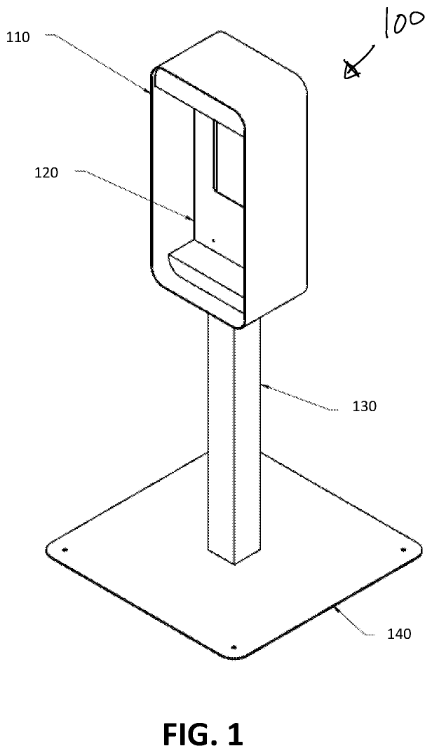

FIG. 1 illustrates a perspective view of an example therapy booth 100. The therapy booth 100 can provide automated mental health care to users who can freely interact with the therapy booth 100. The therapy booth 100 can be placed in city areas in an attempt to help the homeless population and others who may lack a sense of connection and wellbeing.

In one example, the therapy booth 100 can include a shroud 110. The shroud 110 can protect a screen of the booth, such as a touchscreen of a tablet, from weather elements and from direct sunlight during most hours of the day. The shroud 110 can be metal, such as aluminum. In general, the shroud 110 can withstand various human impacts to extend the life of the therapy booth 100.

An insert 120 can lock into place within the shroud 110. In one example, the shroud 110 has an open front face that the insert 120 can be recessed into. The insert 120 can lock into place within the shroud 110. In one example, the locking mechanisms are coupled to a back face of the insert 120 and can mate with additional locking mechanisms inside the shroud 110. For example, a motorized lock can exist on either the back side of the insert 120 or the inside of the shroud 110. The lock can be controlled by a tablet or other touchscreen that is included with the therapy booth 100. For example, when an administrative user logs into the therapy booth 100, the GUI can include an option to unlock the insert 120. This can allow for maintenance, such as swapping a tablet, performing software updates, or uploading new experience clips.

The shroud 110 can alternatively unlock from the backside for administrative access. In one example, the shroud 110 can be coupled to a post 130 that places the shroud 110 high enough in the air for natural human interaction. The post 130 can be made of metal, such as steel or aluminum.

In one example, the post 130 includes a mechanism for height adjustment. For example, a small level on a side of the post 130 can be used to ratchet the shroud 110 higher or lower relative to a baseplate 140. This can allow for people of different heights to adjust the therapy booth 100 for an optimal experience. The adjustment can also be motorized and activated from the GUI, in an example. For example, a motor inside of the post 130 can be activated to raise or lower the shroud 110.

A baseplate 140 can be coupled to the other side of the post 130. Wiring, such as power and ethernet, can run up through the baseplate 140 and post 130 and into the shroud 110 behind the insert 120.

A photovoltaic solar cell can be placed on top of the shroud 110 in an example. This can slowly charge the booth in locations that do not have access to wired power.

In one example, the therapy booth 100 can resemble a phone booth.

The insert 110 can have one or more windows that allow for lights, camera operation, and GUI display. In one example, a touchscreen is provided in a main window. The touchscreen can be a tablet that is attached to the backside of the insert 120.

The tablet can display visuals while speakers directionally output audio towards a user standing in front of the opening of the shroud 110.

When a user is detected by cameras in the therapy booth 100, the GUI can present imagery to capture the user's attention, such as a welcoming message. An introduction clip can play if the therapy booth 100 detects that the user has stopped in front of the therapy booth 100 and is looking at the GUI.

The GUI can present a slider that allows the user to quickly represent their current hope level. For example, the GUI can prompt “please select your current hope level” and the slider can range from 1 to 10. Different experience clips (with different messages) and help options can be presented to the user based on the slider position. For example, a slider level of two can cause video to play that is intended for someone that may be at risk of suicide. Additionally, help services and locations for such a person can be presented at the end of the video. Conversely, a slider level of 8 can cause a video to play that is intended for someone who is relatively stable and happy, yet still needs to be acknowledged and seen.

In this way, the therapy booth 100 can select one or more experience clips to attempt to improve the mental state of the user. In one example, the user can provide additional inputs on the GUI so that the therapy booth 100 can classify how the user is feeling. For example, if the user selects a slow slider position such as 1, the therapy booth 100 can prompt the user regarding whether they need immediate assistance. In one example, the user can also speak to the therapy booth 100. The therapy booth 100 can then display a map with social services that relate to the user inputs. For example, if the user is feeling lonely, they can be directed to an event for meeting others and sharing a meal. Alternatively, if the user is in need of shelter, the map displayed by the therapy booth 100 can lead the user to shelter.

In one example, when the user selects a support service or system, a rideshare program can be notified and the user can be picked up. For example, an UBER or LYFT integration can be used to hail a car to bring the user to the social services destination. Alternatively, government ridesharing can be provided, such as in a police care or ambulance.

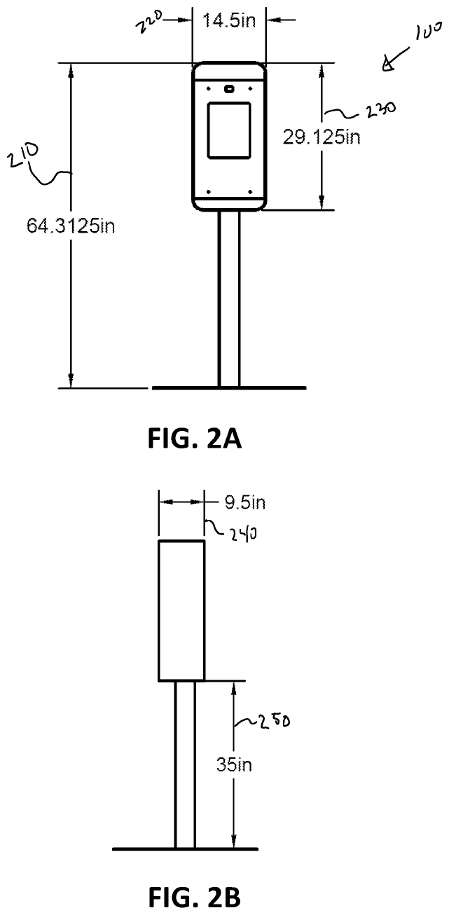

FIG. 2A illustrates a front view of an example therapy booth 100. Example dimensions are shown. For example, the therapy booth 100 height 210 is around 64 inches. The height 210 can be between 55 and 70 inches in another example, with an adjustable post 130.

The width 220 of the therapy booth 100 can be between 12 inches and 16 inches, depending on the embodiment. The shroud 110 height 230 can be between 20 and 40 inches tall. In this example, the shroud 110 height 230 is around 29 inches.

The insert holds the touchscreen at least 5 inches above a bottom side of the shroud.

FIG. 2B illustrates a side view of an example therapy booth 100. In this example, the depth 240 of the shroud can be around 9.5 inches.

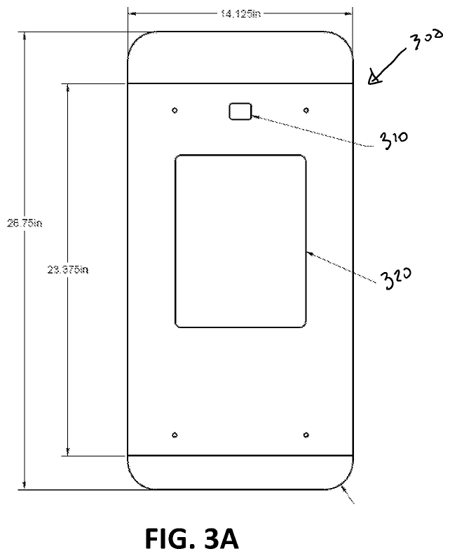

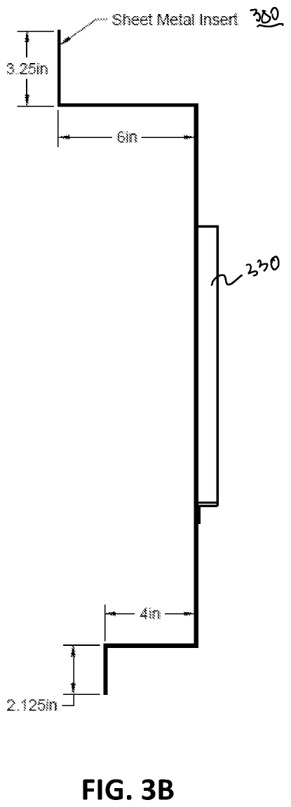

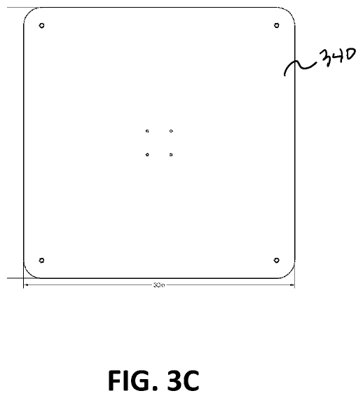

FIG. 3A illustrates a front view of an example insert 300 for a therapy booth 100. The insert 300 can include a first window 320 for accessing a tablet that is secured to the back of the insert 300. A second window 310 can allow a camera to be secured to the back of the insert 300. The camera can detect the user and recognize facial features of the user in one example. Example dimensions are shown in FIG. 3A for the height and width of features of the insert 300.

FIG. 3B illustrates a side view of an example insert 300 for a therapy booth 100. In this view, a tablet 300 is shown as attached to a back side of the insert 300.

FIG. 3C illustrates a top view of an example base plate 340 for a therapy booth 100. The base plate 340 can be secured to the ground in one example. The base plate 340 can be rectangular, square, round, or another shape. In this example, the base plate 340 is a square with a side dimension of about 30 inches.

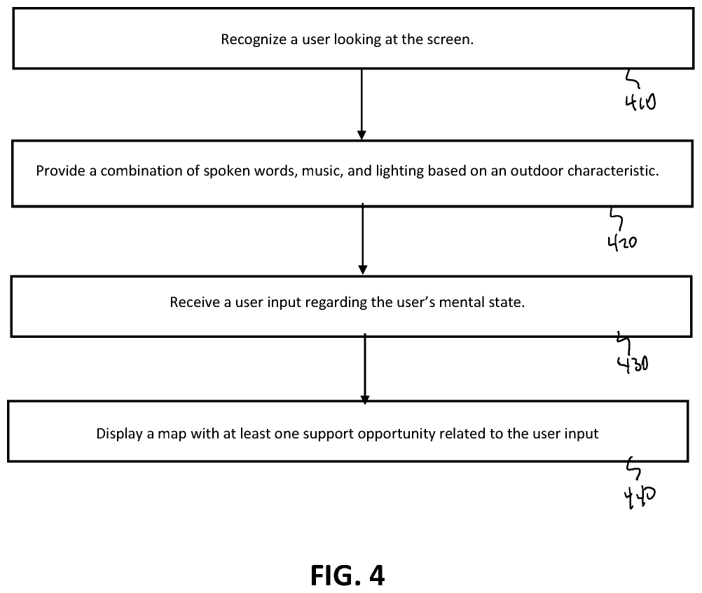

FIG. 4 is an example flowchart of steps performed by a therapy booth 100. At stage 410, the therapy booth 100 can recognize a user looking at the screen. This can include noticing movement and detecting that a user has stopped in front of the therapy booth 100. In one example, the therapy booth 100 can use facial recognition to distinguish the presence of a human who is looking at the tablet. For example, the tablet itself can have a processor that performs the facial recognition.

In one example, user profiles with facial features are stored. This can allow the therapy booth 100 to access past interactions with the same user. For example, the therapy booth 100 can retrieve the user's former hope ratings, display prior therapy service recommendations, and ask the user which of the recommended therapy services the user has previously utilized. This can also allow a user to quickly access the recommend therapy services without sitting through an experience clip again. For example, the user might have tried a first service but be interested in trying a different one that was previously recommended.

Stages 420 and 430 can occur in the order shown in FIG. 4 or in a reverse order. At stage 430, the GUI can receive a user input regarding the user's mental state. This can include displaying a hope slider that the user can manipulate to rank their current hope level, such as from 1 to 10. The user can also indicate whether they are more or less hopeful today than yesterday. This can give the therapy booth 100 some indication regarding the user's improving or declining mental state.

At stage 420, the therapy booth can select an experience clip based on the user's inputs. The experience clip can include both video and sound, such as spoken word. In one example, multiple different experience clips can relate to the user's selected hope level. The use can choose between those, in an example, to prevent the user from having to rewatch the same clip unless they would like to. In another example, the therapy booth 100 tracks which experience clips have been show to the user and attempts to present one that the user has not seen yet.

Additionally, an outdoor characteristic, such as date in the year (i.e., different seasons) and time of day can be used to select the appropriate experience clip, music, and lighting. For example, during winter, the therapy booth 100 can act as a light box that emanates light frequencies that help a person feel less depressed. Additionally, the selected clip can match up with the time of day, such that the person talking in the clip can say “Good morning,” “Good evening,” and other similar conversational characteristics of that time of day and/or season of year. As another example, the person in the video can be dressed according to the outside temperature. This can provide a more realistic interaction for the user.

At stage 440, the therapy booth 100 can display a list of health services that can apply to the user based on the user inputs. For example, a map with locations of health services can be displayed. The user can touch particular services and get more information about the selected service, in an example. These support opportunities can include homeless shelters, a suicide prevention hotline, counselor services, and a hospital, in an example.

The support opportunities can also vary based on placement location of the therapy booth 100. For example, if the therapy booth 100 is placed inside a prison, the support opportunities can include requests for particular services offered by the prison. Additionally, the prison itself can correlate the selected support opportunity to the user by facial recognition or by recognizing a number on the user's uniform.

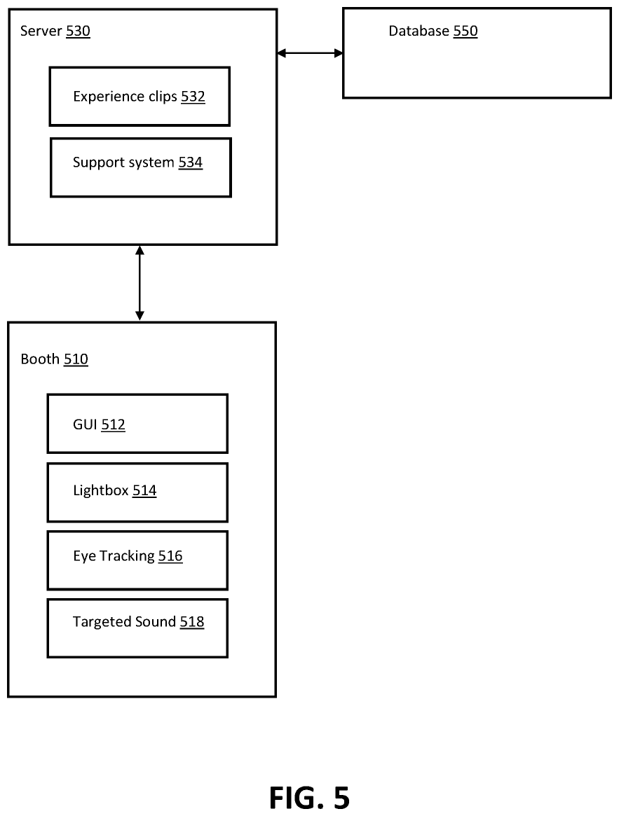

FIG. 5 is an exemplary illustration of system components for providing interactive user therapy. A server 530 can store experience clips 532 and support opportunity information as part of a support system 534. The server 530 can be one or more computers or processor enabled devices. The server 530 can operate as a virtual service in the cloud in one example. The experience clips 532, support system 534 information, and user profiles can be stored in one or more databases 550. The databases 550 can be accessible by the server 530 or part of the server 530.

A therapy booth 510 can be a physical structure placed outdoors in an city area or inside of a facility, such as in a prison. The therapy booth 510 can provide a GUI 512 that allows the user to indicate their current hope level. The therapy booth 510 can be updated to include experience clips 532, support opportunities, and user profiles from the server 530. This can be done automatically, such as by allowing the therapy booth 510 to connect to the server 530 periodically. Alternatively, it can be done manually, such as by loading new information into the therapy booth 510 with a flash drive or a new insert. In one example, the flash drive remains with the tablet on the backside of the insert.

The therapy booth 510 can include a GUI 512, which can be a touchscreen. The GUI can be protected with a screen protector that still allows for interaction but provides additional defense against abuse. In one example, the tablet can detect such abuse, such as detecting movement of the tablet itself. This can cause the camera to record the user interaction for use in catching a vandal.

The GUI 512 can receive user interactions, such as a hope rating on a hope slider. Then the GUI 512 can present one or more corresponding clips that address a user with a corresponding hope rating. The GUI 512 can also present support opportunities that match the user's hope level and location.

The therapy booth 510 can also include a lightbox 514. The lightbox 514 can emit light frequencies that sooth the user and can treat seasonal depression.

The therapy booth 510 can also use one or more cameras for eye tracking. This can ensure that the user is actually still watching or otherwise interacting with the therapy booth 510. If the user stops watching or interacting for a threshold period of time, such as 5 seconds, then the experience clip can stop playing. The therapy booth 510 can detect if the user looks back at the screen and can cause the experience clip to resume. If the user instead walks away, then the therapy booth 510 can display a welcome screen and wait until a new user is detected.

The therapy booth can also provide targeted sound 518. This can include two speakers that aim an optimal distance from the therapy booth, such as two to three feet, to help ensure that the user stands at a proper location and can hear the therapy booth 510 over background noise.

Other embodiments of the invention will be apparent to those skilled in the art from consideration of the specification and practice of the invention disclosed herein. It is understood that the examples can operate as an application or plugin with REVIT or any other BIM or CAD program. Also, the terms part, component, and assembly are used interchangeably. It is intended that the specification and examples be considered as exemplary only, with a true scope and spirit of the invention being indicated by the following claims.

What is claimed is:

1. An interactive therapy booth, comprising:
a shroud having an open front and at least three sides;
an insert that detachably couples to the shroud such that it is set back within the opening, the insert comprising a touchscreen; and
a hardware-based processor that executes the instructions to carry out stages comprising:
recognizing a user looking at the touchscreen;
providing a combination of messages, music, and lighting based on an environmental characteristic and the user being present;
receiving user input on the touchscreen, the input describing a mental state of the user; and
displaying a map on the touchscreen, the map identifying at least one support opportunity related to the user input.

2. The interactive therapy booth of claim 1, wherein the shroud is coupled to a post, and wherein the post is coupled to a base plate, the base plate being attachable to the ground.

3. The interactive therapy booth of claim 1, the stages further comprising:
retrieving a profile associated with the user, the profile indicating prior experience clips seen by the user;
wherein recognizing the user includes fingerprint identification; and
wherein the combination includes an experience clip that has not yet been seen by the user.

4. The interactive therapy booth of claim 1, wherein the environmental characteristic includes time of day, day of the year, outside lighting, and outside temperature.

5. The interactive therapy booth of claim 4, wherein the outside characteristic causes activation of a lightbox therapy.

6. The interactive therapy booth of claim 1, wherein the stages further comprise tracking eyes of the user and changing the combination based on where the user is looking.

7. The interactive therapy booth of claim 1, wherein the stages further comprise ending the message and music based on determining that the user is no longer in front of the interactive therapy booth.

8. The interactive therapy booth of claim 1, further comprising a post coupled to a base plate, wherein the post has an adjustable height.

9. The interactive therapy booth of claim 1, wherein the insert is unlocked based on detecting the fingerprint of an administrative user assigned to the interactive therapy booth.

10. The interactive therapy booth of claim 1, wherein the stages further comprise contacting a server over a cellular network, wherein a plurality of experience clips are downloaded to the therapy booth, wherein the combination includes at least one of the experience clips.

11. The interactive therapy booth of claim 1, the stages further comprising a cleaning element that automatically cleans the touchscreen.

12. The interactive therapy booth of claim 11, the stages further comprising automatically notifying an administrative user regarding at least one of a need to replace the cleaning element and an error with the GUI.

13. The interactive therapy booth of claim 1, wherein the shroud is between 12 inches and 16 inches wide, between 20 and 40 inches tall, and wherein the insert holds the touchscreen at least 5 inches above a bottom side of the shroud.

14. The interactive therapy booth of claim 1, wherein the shroud is coupled to a post, and wherein the post is coupled to a base plate, and wherein a top side of the shroud is at least 5 feet above the base plate.

15. The interactive therapy booth of claim 1, wherein the insert includes an opening for a camera that tracks user eye movement.

16. The interactive therapy booth of claim 1, wherein the insert includes a recessed front surface, wherein the touchscreen is behind the recessed front surface.

17. A method for interactive user therapy, comprising:
recognizing a user looking at the touchscreen, including tracking user eye movement using a camera that is positioned within a booth, the booth having a shroud with an open face, an insert recessed within the shroud, and a window in the insert through which the camera tracks the user eye movement;
receiving user input on the touchscreen, the input describing a mental state of the user, the user input including manipulating a slider that is displayed on the touchscreen to indicate a current hope level of the user;
playing a first experience clip at the booth based on the slider manipulation; and
displaying a map on the touchscreen, the map identifying at least one support opportunity related to the user input.

18. The method of claim 17, further comprising downloading multiple experience clips from a server, wherein the first experience clip is selected based on at least one user selection and the environmental characteristic, the environmental characteristic including at least two of time of day, day of the year, outside lighting, and outside temperature.

19. The method of claim 17, further comprising providing lightbox therapy based on detecting that an outside temperature is below 60 degrees Fahrenheit.

20. The method of claim 17, further comprising:
detecting that the user looks away from the touchscreen;
stopping playing of the first experience clip;
detecting that the user looks back at the touchscreen; and
resuming playing of the first experience clip.
